(19) United States
(12) Patent Application Publication
(10) Pub. No.: US 2013/0046181 A1
(43) Pub. Date: Feb. 21, 2013

(54) Systems And Methods For Detecting Airway Occlusion

(71) Applicants: Mohammad Al-Abed, Arlington, TX (US); Khosrow Behbehani, Arlington, TX (US); Pietro Antich, Richardson, TX (US); Donald Watenpaugh, (US); John Burk, (US)

(72) Inventors: Mohammad Al-Abed, Arlington, TX (US); Khosrow Behbehani, Arlington, TX (US); Pietro Antich, Richardson, TX (US); Donald Watenpaugh, (US); John Burk, (US)

(73) Assignee: Board of Regents, The University of Texas System, Austin, TX (US)

(21) Appl. No.: 13/588,819

(22) Filed: Aug. 17, 2012

Related U.S. Application Data
(60) Provisional application No. 61/524,403, filed on Aug. 17, 2011.

(51) Int. Cl. A61B 8/08

(52) U.S. Cl. 600/442

(57) ABSTRACT

In one embodiment, airway occlusion detection is performed by applying ultrasonic pulses into the neck of a patient, receiving the ultrasonic pulses after they have passed through the neck, and analyzing the pulses to determine whether or not an airway of the patient is partially or fully occluded.

CROSS-REFERENCE TO RELATED APPLICATION(S)

This application claims priority to co-pending U.S. Provisional Application Ser. No. 61/524,403, filed Aug. 17, 2011, which is hereby incorporated by reference herein in its entirety.

### BACKGROUND

Obstructive sleep apnea/hypopnea (OSAH) is the most common form of sleep disordered breathing (SDB). It is defined by repetitive pharyngeal airway collapse or narrowing for ten seconds or longer, occurring five times or more per hour. OSAH is prevalent in up to 15% of the middle age population. Recent epidemiological studies have showed high prevalence of the disorder in both males and females, contrary to the long held belief that OSAH was mostly prevalent in males.

OSAH patients have a high risk of hypertension and adverse cardiovascular implications, along with an array of major co-morbidities including daytime sleepiness, poor quality of life, depression, and cognitive impairment. Risk of automobile accidents for OSAH patients have been reported to be seven fold that of the average. Retrospective studies show that there is an association between OSAH and morbidity and mortality due to its cardiovascular and cerebrovascular implications. Along with daytime sleepiness, patients with OSAH suffer from some or all of the following: sleep fragmentation, habitual snoring, morning headaches, and systemic hypertension.

Nocturnal polysomnography (NPSG) is the current gold standard for diagnosis of OSAH. NPSG is an overnight study in which multiple physiological markers are collected that aid in identifying the quality of sleep, the presence of OSAH events, and their prevalence. Most commonly, such markers include electroencephalography (EEG), electrooculography (EOG), chin and leg electromyography (EMG), electrocardiography (ECG or EKG), arterial oxygen saturation (SaO₂), snoring and nasal airflow, and chest and abdominal movement. Using the recorded signals, a sleep technician manually identifies the sleep stages, apnea/hypopnea events, and periodic limb movements. The technician then presents his or her findings to a physician specialized in sleep medicine, who recommends the type of treatment based on the severity of the disorder and the medical history of the patient. While NPSG can be effective, it is costly because of the use of multiple instruments, multiple night studies, and the labor cost associated with patient instrumentation, observation, and review of records.

In view of the prevalence of OSAH, its socioeconomic cost, the limited number of credited sleep labs, and the high cost of a diagnostics sleep study, it can be appreciated that it would be desirable to have alternative OSAH screening systems and methods.

### BRIEF DESCRIPTION OF THE DRAWINGS

The present disclosure may be better understood with reference to the following figures. Matching reference numerals designate corresponding parts throughout the figures, which are not necessarily drawn to scale.

FIG. 1 is a schematic view of an embodiment of a system for detecting airway occlusion.

FIG. 2 is a schematic view of an embodiment of a patient interface used in the system of FIG. 1.

FIG. 3 is a block diagram of an embodiment of a control unit used in the system of FIG. 1.

FIG. 4 is a schematic view of a further embodiment of a system for detecting upper airway occlusion.

FIG. 5 is a block diagram of a patient interface used in the system of FIG. 4.

FIG. 6 is a block diagram of an embodiment of a computing device that can be used in the systems of FIGS. 1 and 4.

FIG. 7 is a flow diagram of an embodiment of a method for detecting airway occlusion.

FIGS. 8A-8B is a flow diagram of an embodiment of a method for analyzing a collected ultrasound signal to identify airway occlusion.

FIG. 9 is a graph of an example received ultrasound signal.

FIG. 10 is a graph of a rectified version of the ultrasound signal of FIG. 9.

FIG. 11 is a graph identifying an envelope of the rectified ultrasound signal of FIG. 10.

FIGS. 12A-12J are graphs that illustrate example features that can be extracted from a received ultrasound signal in analyzing a received ultrasound signal.

### DETAILED DESCRIPTION

As described above, it would be desirable to have obstructive sleep apnea/hypopnea (OSAH) screening systems and methods that avoid the drawbacks associated with current systems and methods. Disclosed herein are systems and methods that use non-invasive ultrasonic sensing to detect partial or full occlusion of a patient airway that is indicative of OSAH. Instead of producing an image of the airway, ultrasound is used to create an ultrasonic signature of the patient's neck that can be analyzed to identify the occlusions. In some embodiments, ultrasonic pulses are applied to the neck and are then received, for example on the opposite side of the neck. The received pulses are stored and analyzed using one or more algorithms that distinguish an open airway from a partially or fully occluded airway based upon certain features extracted from the received pulses. A sleep disordered breathing (SDB) diagnosis can then be made based upon the frequency and severity of the occlusions.

In the following disclosure, various specific embodiments are described. It is to be understood that those embodiments are example implementations of the disclosed inventions and that alternative embodiments are possible. All such embodiments are intended to fall within the scope of this disclosure.

OSAH is classified as a sleep disorder under the ICD-10 (International Statistical Classification of Diseases, 10th Revision), which is a subgroup of the episodic and paroxysmal disorders, which is in turn a group of the diseases of the nervous system. It is defined in adults as five or more episodes per hour of cessation of breathing (apnea) or 50% or more partial airway occlusions (hypopneas) for ten seconds or more. By definition, the patient continues the respiratory effort and movements during these events, and a cyclic recurrence of these episodes occurs during the night. The length of each episode is typically 20-40 seconds, with some rare severe cases extending more than 60-90 seconds. Snoring is often an associated characteristic of the disorder, along with cyanosis and whole body movements occurring at the arousal.

The underlying causes of OSAH may be different for different patients, and are related to the upper airway anatomy, obesity, airway muscular and neurological activity, arousal threshold, chemoreflex sensitivity, and complex mechanisms that contribute to all or some of these underlying causes. An anatomically small pharyngeal airway is one of the major underlying causes of OSAH. This is due abnormalities in the skull bone structure, and mandible location compared to the maxilla and the hard palate. Other soft tissue abnormalities, such as an increase in the volume of the tongue, the soft palate, the parapharyngeal fat pads (especially in cases of obesity), and the lateral walls surrounding the pharynx, are associated with cases of OSAH. However, anatomical abnormality only constitutes one-third of all cases of OSAH.

The upper airway is kept patent (open) by the activity of the upper airway dilator muscles: Levator veli palatine, tensor veli palatine, palatoglossus, palatopharyngeus and the genioglossus. The activation of these muscles is thought to be higher in patients with OSAH compared to normal subjects during wakeful hours. On the onset of sleep, there is a general decrease in the muscle tone (hypotonia). This causes a slight increase in the airway resistance in normal subjects, whereas it causes a large increase in airway resistance in the OSAH patients, who inherently have a smaller airway volume. The activation of the pharyngeal dilators is known as the upper airway reflex, and its purpose is to force the upper airway to stay open to counter act the negative airway pressure during inhalation.

The upper airway is thought to be at most risk of collapse during the time after expiration and before inspiration (when the pharyngeal dilators receive the signals to contract). However, for OSAH patients, several factors cause the central respirator centers to fail to increase the muscle tone of the pharyngeal dilators before the onset of arousal. These factors include impaired sensory information from the upper airway, decreased cortical arousal threshold, or an increased loop gain in the motor control of ventilatory stability. The failure of the central respirator centers to increase the muscle tone is further amplified and is more prevalent during rapid-eye movement (REM) sleep.

The site of obstruction of the airway during apnea events differs, with intra- and inter-subject variations, and with variation in the obstruction site between sitting and supine positions. However, most of the studies, irrespective of the technique to localize the occlusion, indicate that the main site of occlusion is the oropharynx, with extension to the laryngopharynx, of the upper airway.

With occlusion of the upper airway being one of the root causes of OSAH, it would be desirable to detect such occlusion as a means of diagnosing OSAH. As described above, upper airway occlusions can be detected by analyzing ultrasonic pulses that have been applied to the neck. Because an ultrasonic signal transmitted through or reflected from an open airway will have different temporal, spatial, and spectral features compared to those associated with a partially or fully occluded airway, the patency of the airway can be determined from the analysis.

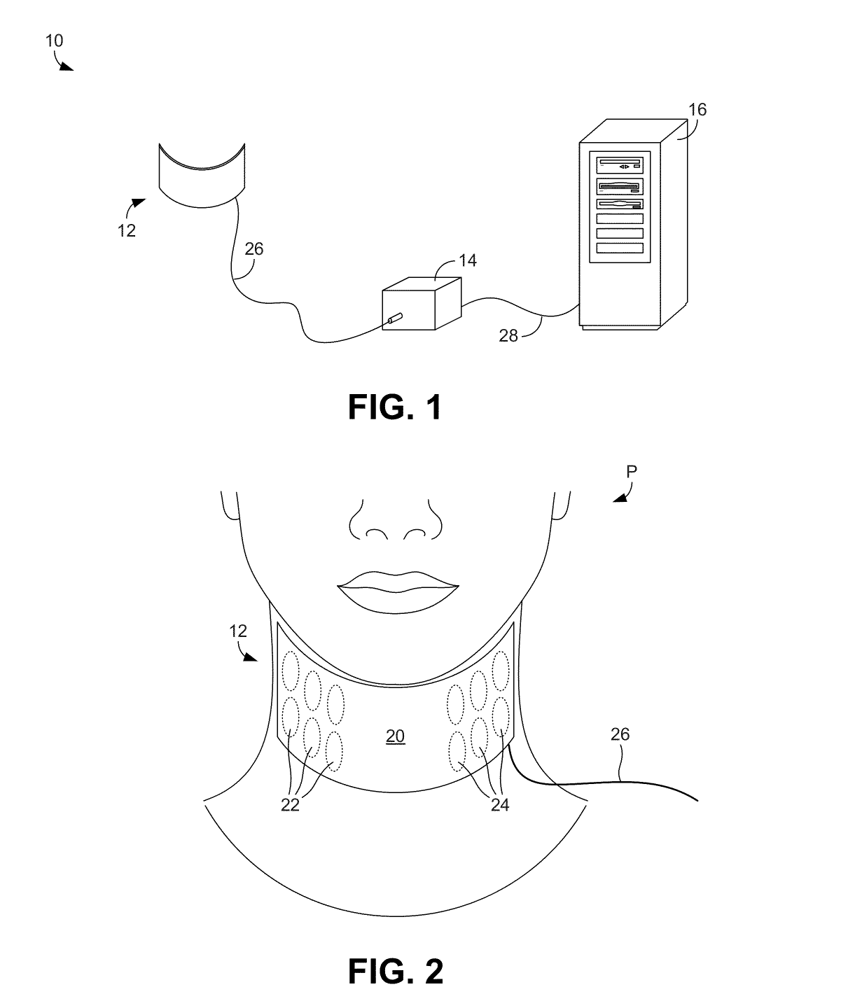

FIG. 1 illustrates a first embodiment of a system 10 for detecting airway occlusion. As shown in that figure, the system 10 generally comprises a patient interface 12, a control unit 14, and a computing device 16. As is described below, the patient interface 12 and the control unit 14 can, in some embodiment, form part of an inexpensive home testing system that can be provided to a patient to take home with them for the purpose of collecting data as they sleep.

The patient interface 12 is a device that can be applied to the exterior of the patient's neck level with his or her upper airway. In some embodiments, the patient interface 12 is configured as a collar that wraps around the patient's neck. In other embodiments, the patient interface 12 can incorporate an adhesive that enables the interface to be adhered to the patient's neck. Irrespective of its specific configuration, the patient interface 12 at least comprises elements that are adapted to transmit and/or receive ultrasonic pulses so that the effects of the patient's upper airway on the pulses can be evaluated.

FIG. 2 illustrates an example embodiment for the patient interface 12, and shows the interface applied to the neck of a patient P. As shown in that figure, the patient interface 12 generally comprises an elongated body 20 that extends from one lateral side of the neck to the opposite lateral side of the neck. Provided within or on an inner surface of the body 20 are one or more transducers that are adapted to transmit and/or receive ultrasonic waves. In the illustrated embodiment, the interface 12 comprises transmitting transducers 22 positioned at one end of the body 20, and receiving transducers 24 positioned at the other end of the body. In such an embodiment, ultrasonic pulses can be applied to the neck from multiple positions on one side of the neck, and then received from multiple positions on the opposite side of the neck. The transducers 22, 24 can take various forms. In some embodiments, the transducers 22, 24 are piezoelectric ceramic transducers, such as polycrystalline lead-zirconate-titanate (PZT) transducers, and have a central resonant frequency of approximately 500 kHz to 10 MHz. In some embodiments, the transducers have a central resonant frequency of approximately 3 MHz.

In the example of FIG. 2, the patient interface 12 comprises a total of twelve transducers, including six transmitting transducers 22 and six receiving transducers 24. Notably, it is not necessary to use multiple transmitting and receiving transducers 22, 24. The use of multiple transducers may be desirable, however, because it increases the likelihood that at least one ultrasound signal will reach the upper airway and be received, either by way of transmission through the neck or reflection from the neck. When multiple receiving transducers 24 are used, multiple ultrasound signals (channels) can be collected and analyzed.

Although separate transmitting and receiving transducers are shown in FIG. 2, it is noted that, in other embodiments, the same transducers can be used to both transmit and receive the ultrasonic pulses. In such a case, the received pulses are those that are reflected back from the features within the neck, including the upper airway. When the transducers are used to both transmit and receive, the interface 12 can comprise transducers on only a single side of the patient's neck or both sides of the neck.

As is further illustrated in FIG. 2, a cable 26 extends from the patient interface 12. As shown in FIG. 1, that cable 26 can be used to link the interface 12 to the control unit 14.

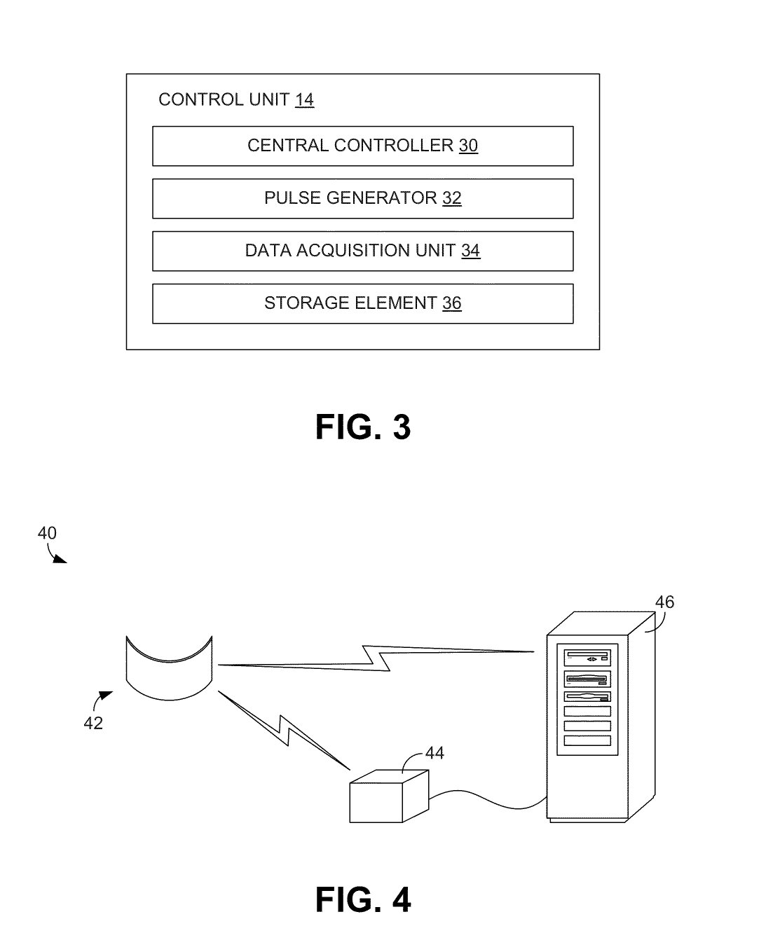

The control unit 14 can be used to control acquisition and storage of data from the patient. FIG. 3 illustrates an example embodiment for the control unit 14. In that embodiment, the control unit 14 includes a central controller 30, such as a processing device, that controls its overall operation, a pulse generator 32 that is configured to generate electrical pulses for delivery to the transmitting transducers of the patient interface 12, and a data acquisition unit 34 that is configured to receive and store the ultrasound signals collected by the receiving transducers of the patient interface. In some embodiments, the control unit 14 can be a portable bedside unit that the patient can activate to initiate and control data collection. As is further shown in FIG. 3, the control unit 14 can include a storage element 36 in which the received ultrasonic signals can be stored.

With reference back to FIG. 1, the computing device 16 can run a software-based SDB diagnosis system that comprises one or more algorithms that are configured to analyze received ultrasound signals to detect partial or full airway occlusions. In the home testing scenario, data collected by the control unit 14 can be downloaded to the computing device 16 using a further cable 28.

FIG. 4 illustrates a second embodiment of a system 40 for detecting airway occlusion. The system 40 is similar in several ways to the system 10 of FIG. 1. Therefore, the system 40 generally comprises a patient interface 42, a control unit 44, and a computing device 46. However, in the embodiment of FIG. 4, the patient interface 42 is configured to wirelessly communicate with the control unit 44. In addition, the patient interface 42 incorporates some of the functionality of the control unit 14 described in relation to FIGS. 1 and 3.

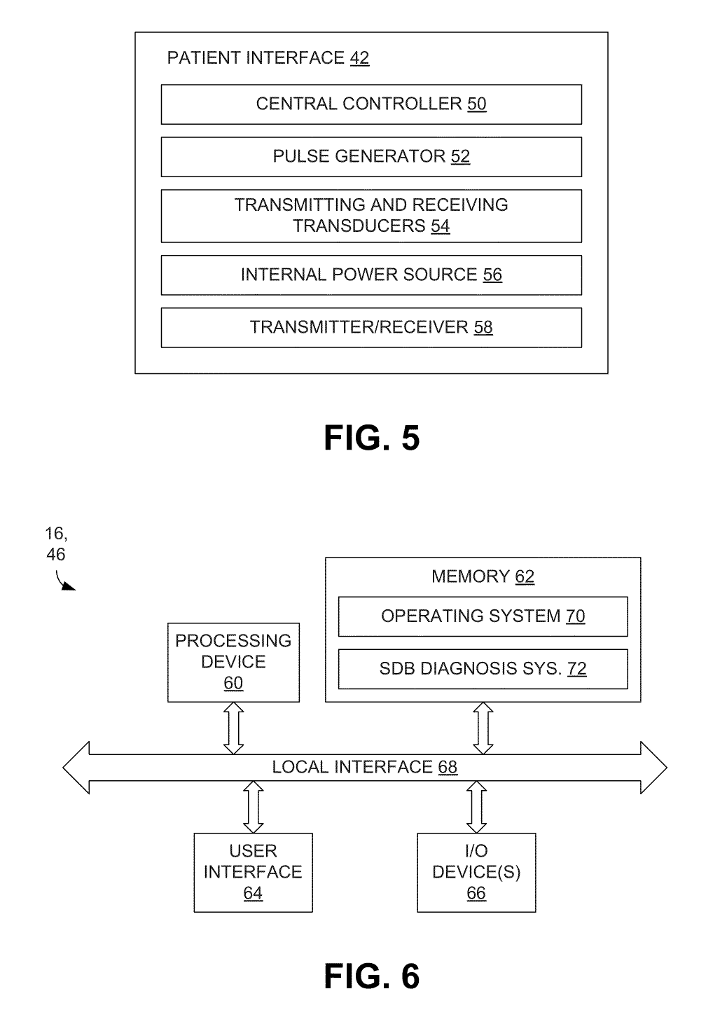

FIG. 5 illustrates an example embodiment for the patient interface 42. In the embodiment of FIG. 5, the patient interface 42 includes transmitting and receiving transducers 54 just like the patient interface 12. In addition, however, the interface 42 includes its own pulse generator 52 that is configured to generate electrical pulses to drive the transmitting transducers. The pulse generator 52 is controlled by a central controller 50 of the interface, such as a microprocessor, and is powered by an internal power source 56, such as a battery. As is further illustrated in FIG. 5, the patient interface 42 also includes a wireless transmitter/receiver 58 that can be used to wirelessly receive commands from the control unit 44 (e.g., a power command and/or a start command) and wirelessly transmit the ultrasound signals received by the receiving transducers. In some embodiments, the wireless communications can be based on WiFi (IEEE 802.11) or Bluetooth (IEEE 802.15).

FIG. 6 illustrates an example embodiment for the computing device 16 shown in FIG. 1 and the computing device 46 shown in FIG. 5. As shown in FIG. 6, the computing device 16, 46 comprises a processing device 60, memory 62, a user interface 64, and at least one I/O device 66, each of which is connected to a local interface 68.

The processing device 60 can include a central processing unit (CPU) or a semiconductor based microprocessor (in the form of a microchip). The memory 62 includes any one of or a combination of volatile memory elements (e.g., RAM) and nonvolatile memory elements (e.g., hard disk, ROM, etc.). The user interface 64 comprises the components with which a user interacts with the computing device 16, 46, and the I/O devices 66 are adapted to facilitate communications with other devices.

The memory 62 is a non-transitory computer-readable medium and stores programs (i.e., logic) including an operating system 70 and an SDB diagnosis system 72. As described above, the SDB diagnosis system 72 includes one or more algorithms that are configured to analyze the collected ultrasound signals and distinguish open airways from partially or fully occluded airways. In some embodiments, features are extracted from the received ultrasonic pulses and those features are evaluated to determine when occlusions occur. A diagnosis can then be made based upon frequency and severity of the occlusions.

Although two specific embodiments of systems have been illustrated and described above, it is noted that other systems for detection of airway occlusion can take alternative forms. For example, in another embodiment, the control unit can communicate the received ultrasound signals to a computing device wirelessly. In a further embodiment, the control and storage functionalities of the control unit can be integrated into the patient interface. In such a case, the received ultrasound signals can be stored on the patient interface for later download to a computing device, or can be transmitted periodically or real time as they are collected. Numerous other variations are possible and are deemed to fall within the scope of this disclosure.

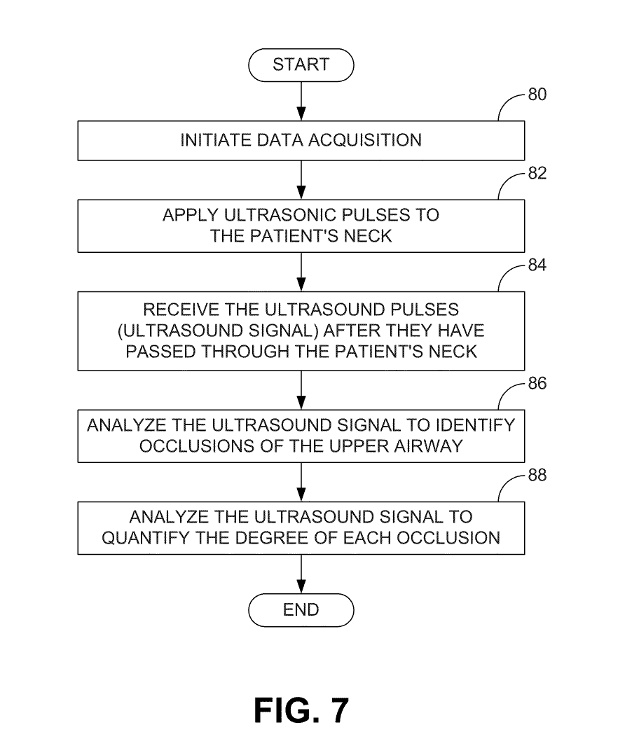

FIG. 7 is a flow diagram that provides an overview of an embodiment for detecting occlusion of a patient's airway for purposes of diagnosing OSAH. Beginning with block 80, data acquisition is initiated. As noted above, the data can, in some embodiments, be acquired in a home testing scenario. Therefore, the patient can, for example, obtain a home OSAH detection kit that includes a patient interface and a control unit. Prior to retiring for the evening, the patient can place the control unit next to his or her bed (e.g., on a nightstand) and don the patient interface, ensuring that the transducers of the interface are placed in the appropriate locations on the neck. The patient can then, for example, initiate data acquisition by pressing an appropriate button provided on the control unit.

Referring next to block 82, ultrasonic pulses are then applied to the patient's neck using the transmitting transducers of the patient interface. In cases in which the control unit comprises the pulse generator, electrical pulses can be transmitted from the control unit to the patient interface via a cable. In cases in which the patient interface comprises the pulse generator, the control unit can instead send a control signal to the interface to command it to commence pulse generation. Irrespective of the location of the pulse generator, the ultrasonic pulses are non-ionizing pulses and therefore have no physiological effect on the patient. The pulses are very short pulses that are emitted with high frequency. In some embodiments, each pulse is approximately 0.5 to 1 microseconds (μs) in duration and a pulse is emitted every 50 to 500 milliseconds (ms). By way of example, some embodiments, approximately 10 pulses are emitted each second.

Once pulses have been applied to the patient's neck, they can be received as part of a continuous ultrasound signal, as indicated in block 84. When the patient interface comprises multiple receiving transducers, multiple independent ultrasound signals can be collected. In some embodiments, the received ultrasound signal(s) can be stored on the control unit. In the home testing scenario, the patient can halt data acquisition upon waking and then return the control device to his or her physician, who can then download the stored ultrasound signal(s) to a computing device for processing. In other embodiments, the patient can download the ultrasound signal(s) to his or her home computing device and transmit the signal(s) to the physician, for example using the Internet.

Regardless of how the ultrasound signal or signals are conveyed, they can then be analyzed to identify occlusions of the upper away, as indicated in block 86. In particular, the changes in the ultrasonic pulses that were applied to the neck are analyzed. Because the amplitude and the frequency of the pulses are changed by the features of the neck, including the upper airway, those changes provide an indication of the status of the airway (i.e., open or partially/fully occluded). When multiple ultrasound signals have been collected, one or more of the channels can be combined (e.g., averaged together) and the analysis can be performed on the combined signal. Alternatively, each individual ultrasound signal can be independently evaluated and the one or more signals that appear to be most sensitive to airway occlusions can be selected for analysis.

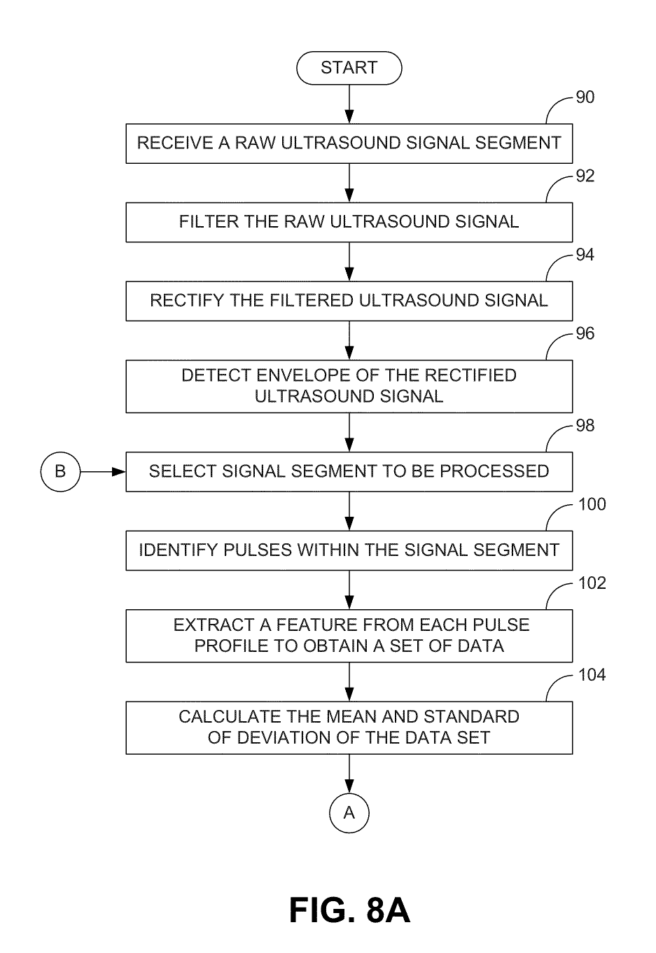

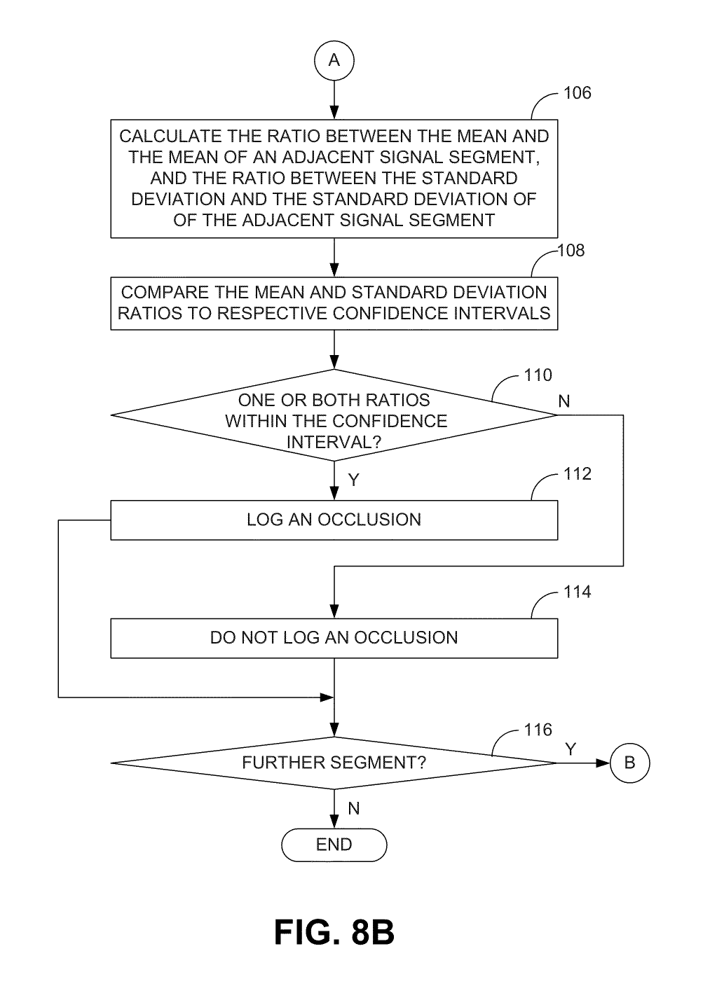

Detailed examples of the analysis performed on the ultrasound signals are described in relation to FIGS. 8-12. As is discussed below, one or more features can be extracted from each received ultrasonic pulse and the occlusion determination can be made based upon those features and how they change over time.

With reference next to block 88 of FIG. 7, the analysis can, in some embodiments, further include analyzing the ultrasound signal to quantify the degree of the occlusions. By way of example, partial occlusions can be quantified in terms of percentage of occlusion. In addition or exception, an apnea/hypopnea index (AHI) can be generated to provide an indication of the severity of the disease.

FIGS. 8A and 8B describe an example embodiment of a method for analyzing a collected ultrasound signal, such as a signal collected by one of the receiving transducers of a patient interface or a combined signal. In some embodiments, at least some of the actions described in FIGS. 8A and 8B are performed on a computing device, for example by the SDB diagnosis system 72. It is noted that the various actions described in relation to FIGS. 8A and 8B do not necessarily need to be performed in the order identified in FIGS. 8A and 8B.

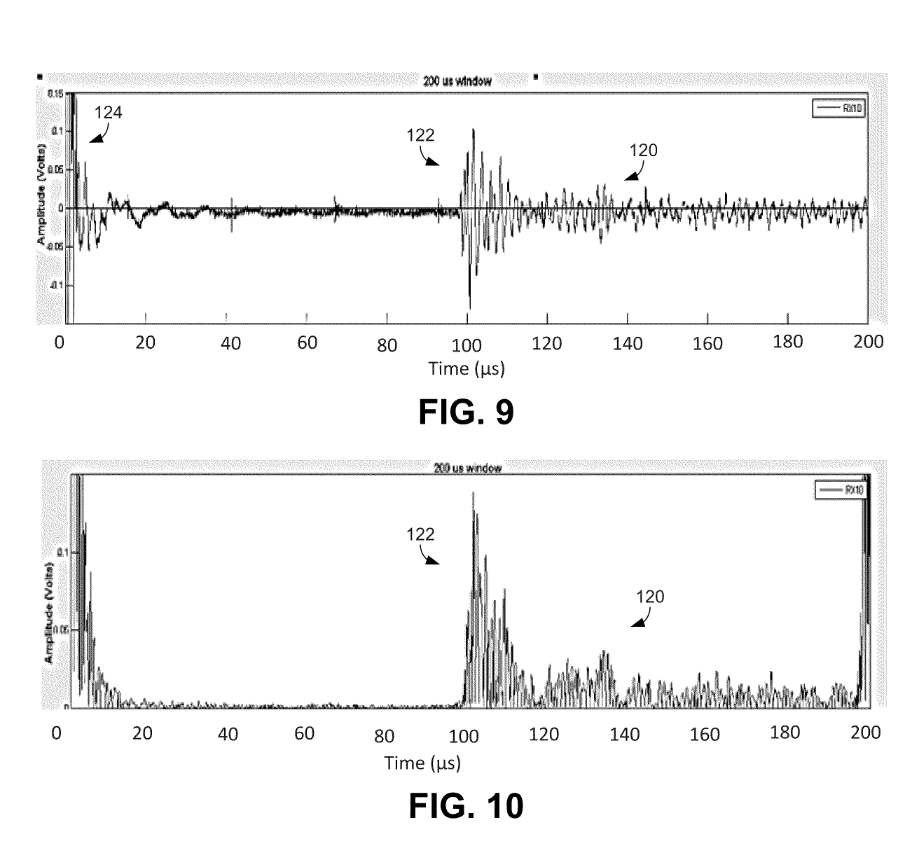

Beginning with block 90 of FIG. 8A, a raw ultrasound signal is received. As described above, the signal can have, for example, been downloaded from a control unit that was used in the patient's home. FIG. 9 illustrates a portion of an example raw ultrasound signal 120. As is indicated in that figure, an ultrasonic pulse 122 appears around 100 μs that is related to an applied pulse 124 that appears just after 0 μs. In some embodiments, the signal 120 can be an amplified signal that was previously amplified, for example by an inline amplifier of the control unit.

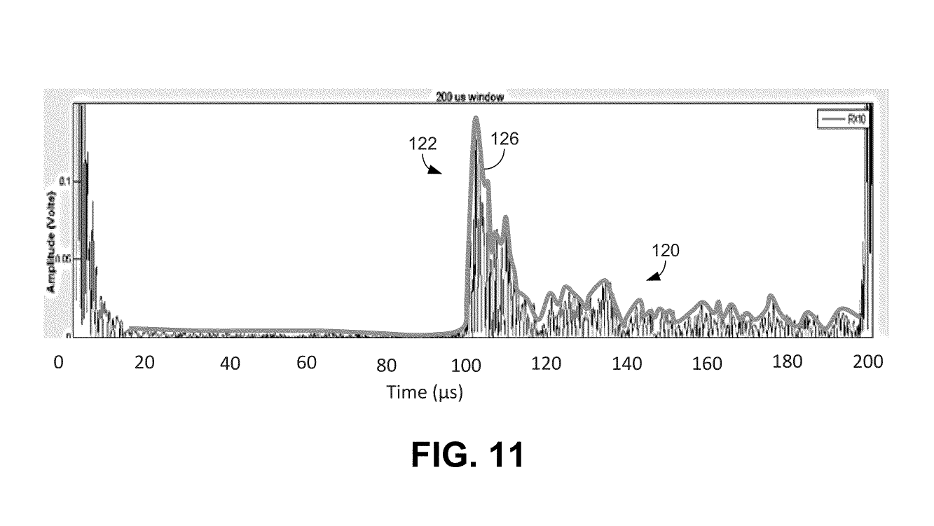

With reference back to FIG. 8A, the raw ultrasound signal can be filtered to remove noise, as indicated in block 92. In some embodiments, the signal can be filtered with a band-pass filter with a low cutoff frequency of approximately 0.15 MHZ and a high cutoff frequency of approximately 2 MHz. Next, with reference to block 94, the filtered signal can be rectified so that the amplitude of the signal is only positive. FIG. 10 illustrates the ultrasound signal 120 after filtering and rectifying. Next, the envelope of the rectified ultrasound signal can be detected, as indicated in block 96 of FIG. 8A, to remove the high-frequency components of the signal. Such envelope detection can, in some embodiments, be performed using cubic spline interpolation. As is shown in FIG. 11, what results from the envelope detection is a curve 126 that can be used in further stages of the analysis.

At this point, one or more features can be extracted from the pulses of the ultrasound signal to make occlusion determinations. In some embodiments, the features of the pulses of discrete segments of the signal are extracted and compared with extracted features of the pulses of other discrete segments of the signal to identify changes over time in the patient's breathing that are indicative of OSAH. Therefore, with reference to block 98 of FIG. 8A, a signal segment to be processed is selected. In some embodiments, the segment spans approximately 0.5 to 30 seconds and will comprise many received pulses. By way of example, the segment can span 10 seconds and comprise 100 such ultrasonic pulses.

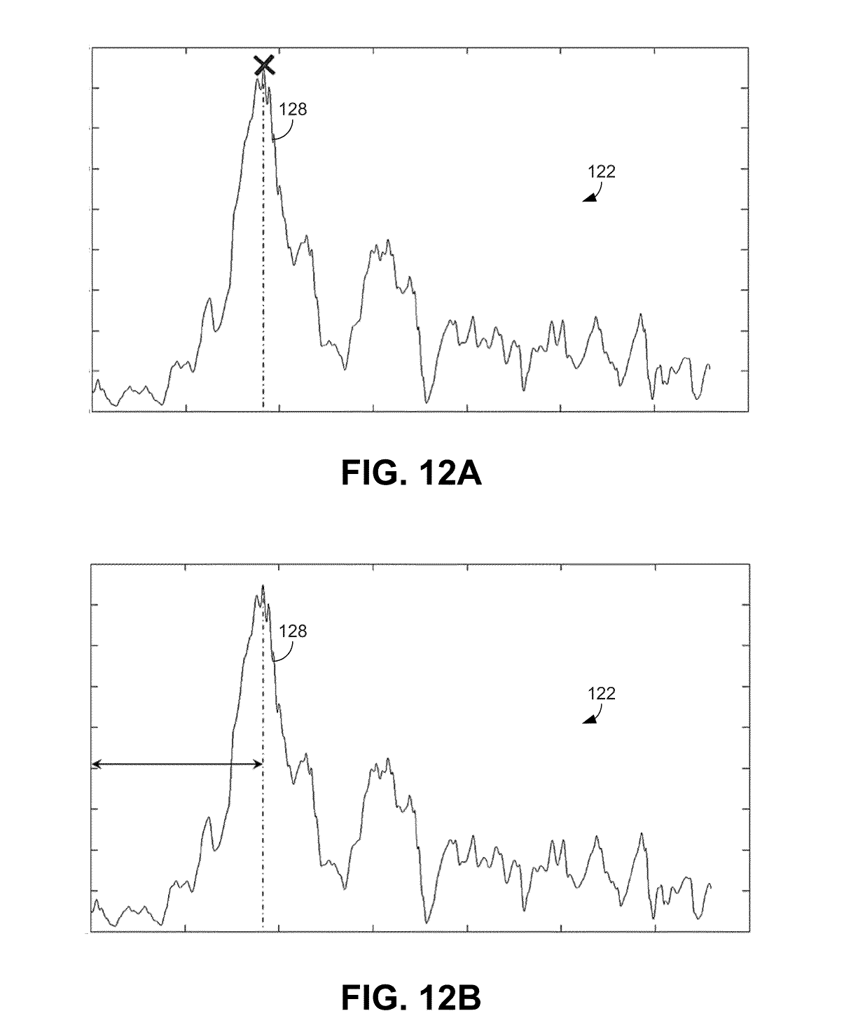

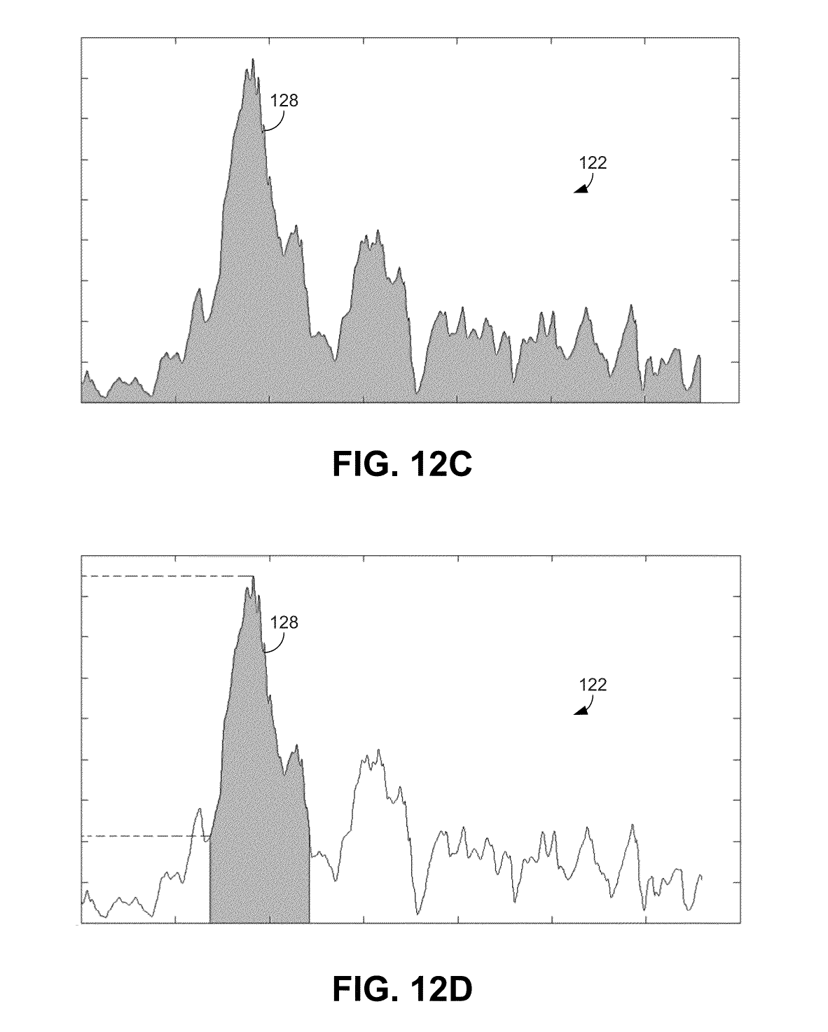

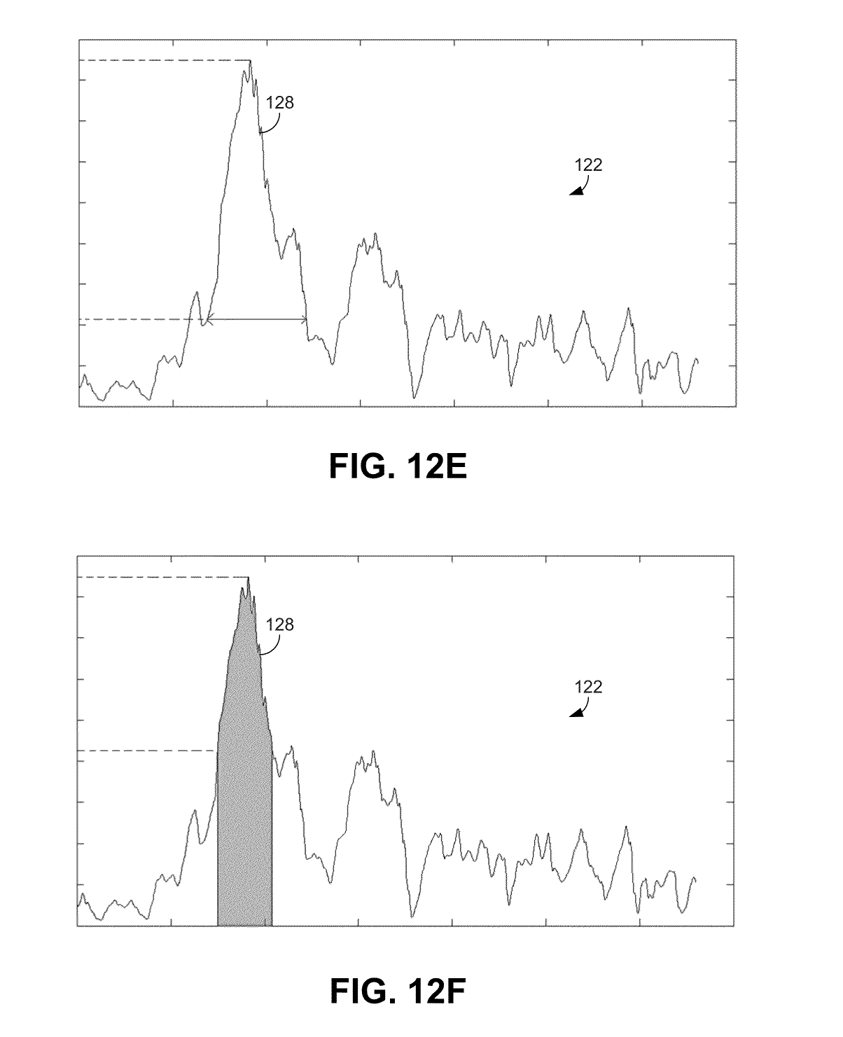

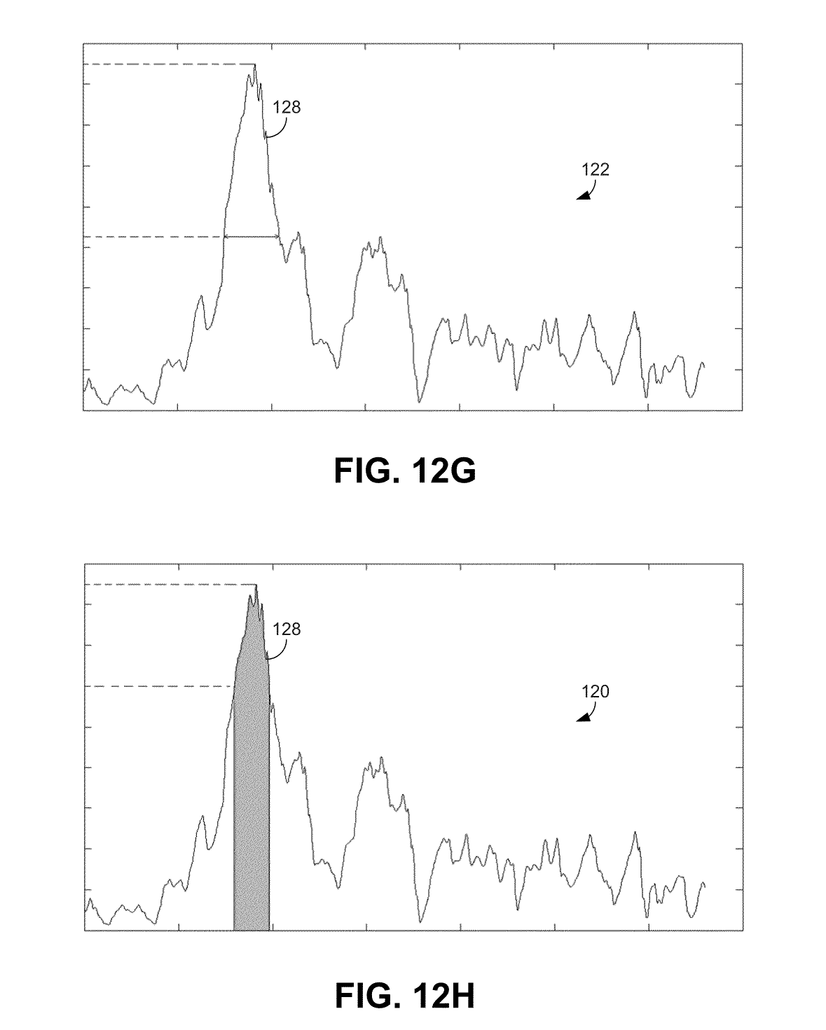

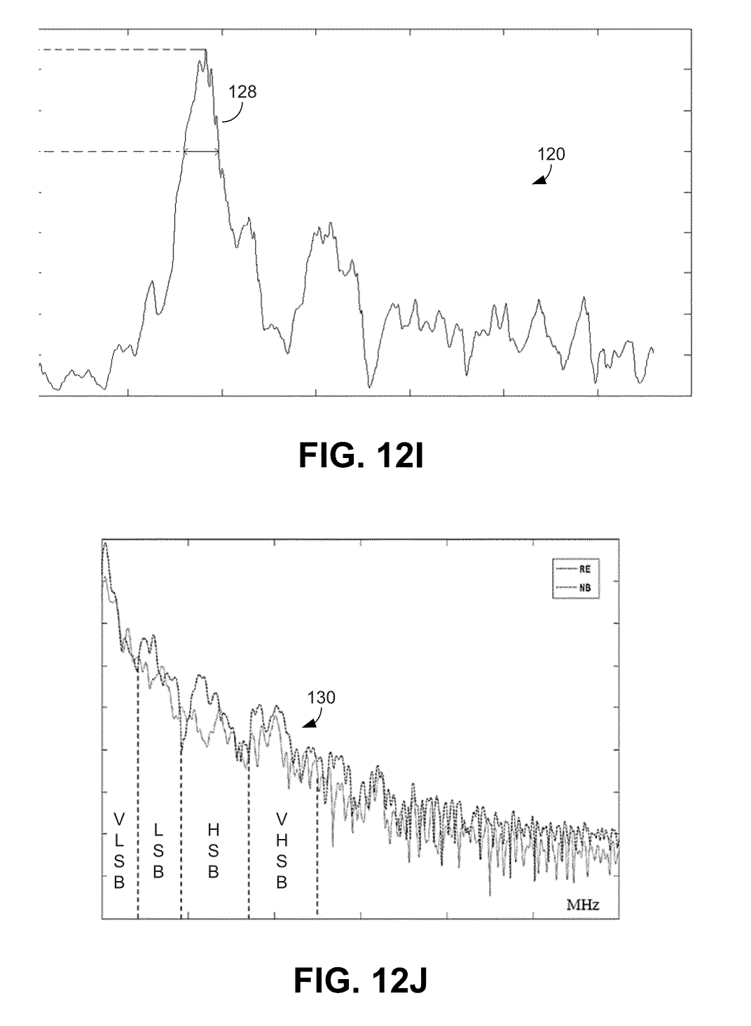

Referring next to block 100, the pulses in the signal segment are identified. For the remainder of the discussion of FIGS. 8A and 8B, the single pulse 122 will be considered for purposes of discussion. Once the pulses have been identified, one or more features of each pulse are extracted, as indicated in block 102. In some embodiments, multiple features are extracted, each pertaining to a different aspect of the pulse. FIGS. 12A-12I illustrate ten temporal features that can be extracted, and FIG. 12J illustrates four spectral features that can be extracted.

A first feature that can be extracted is the peak of the pulse. As is shown in FIG. 12A, the peak of the pulse profile 128 is the peak voltage observed for the pulse. A second feature that can be extracted is the location of the peak of the pulse. As is shown in FIG. 12B, the peak location is identified by the time at which the peak voltage occurred.

A third feature that can be extracted is the total area under the pulse profile. That area is identified with shading in FIG. 12C. A fourth feature that can be extracted is the area under the pulse profile associated with 25% of the peak amplitude. As is shown in FIG. 12D, that area is the area under the pulse profile 128 between the two times at which the profile crosses the 25% point. A fifth feature that can be extracted is the time span associated with 25% of the peak amplitude. As is shown in FIG. 12E, that time span is that which extends between the two times at which the pulse profile 128 crosses the 25% point.

A sixth feature that can be extracted is area under the pulse profile associated with 50% of the peak amplitude. As is shown in FIG. 12F, that area is the area under the pulse profile 128 between the two times at which the profile crosses the 50% point. A seventh feature that can be extracted is the time span associated with 50% of the peak amplitude. As is shown in FIG. 12G, the time span that extends between the two times at which the pulse profile 128 crosses the 50% point. An eighth feature that can be extracted is the area under the pulse profile associated with 70% of the peak amplitude. As is shown in FIG. 12H, that area is the area under the pulse profile 128 between the two times at which the profile crosses the 50% point. A seventh feature that can be extracted is the time span associated with 70% of the peak amplitude. As is shown in FIG. 12I, that time span is that which extends between the two times at which the pulse profile 128 crosses the 70% point.

Referring next to FIG. 12J, illustrated are four spectral features in the form of the areas under the curve for four spectral bands, including a very low spectral band (VLSB), a low spectral band (LSB), a high spectral band (HSB), and a very high spectral band (VHSB). In some embodiments, the LFSB includes the 10-230 kHz frequency range, the LSB includes the 230-470 kHz frequency range, the HSB includes the 470-770 kHz frequency range, and the HFSB includes the 0.77-1.22 MHz frequency range. The area under the curve for each of those bands can be calculated by first performing a Fourier transform of the pulse profile to obtain a spectrum 130 in the frequency domain, and then calculating the area under the curve between the boundaries of each frequency band.

Although particular features have been described above, it is noted that other features could be extracted, if desired. Moreover, it is noted that not all of the features must be extracted. Instead, one or several of the features described above can be used. Regardless, once all of the features that are to be extracted have been extracted from each pulse of the segment, the mean and standard deviation of those features can be calculated, as indicated in block 104. For example, if the signal segment spanned 10 seconds and contained 100 pulses, 100 peak values could exist that could be used to calculate a mean and standard deviation.

Referring next to block 106 of FIG. 8B, the ratio between the calculated mean and the mean of an adjacent signal segment is calculated, as is the ratio between the calculated standard deviation and the standard deviation of the adjacent signal segment. By calculating these ratios, changes in the pulses (and therefore the breathing of the patient) between the two adjacent time segments can be identified. Such a ratio should include 1 if no change in the airway has occurred. Using these ratios, one can detect whether or not a partial or full airway occlusion has occurred. By way of example, ratios of the means or standard deviations that are associated with known transitions from normal respiration to respiratory (e.g., OSAH) events can be pooled to form a statistically-significant confidence interval at a desired level (e.g., 95%). Then, the mean and/or standard deviation ratios of a given feature computed for two adjacent signal segments can be compared with their respective confidence intervals, as indicated in block 108, and if a ratio lies within the confidence interval, an airway occlusion has occurred with a given degree of certainty (e.g., 95%). Therefore, with reference to decision block 110, if one or both of the ratios are within their respective confidence interval, a change in breathing has occurred and an occlusion is logged, as indicated in block 112. If, on the other hand, neither of the ratios is within their respective confidence intervals, no occlusion is logged, as indicated in block 114. In cases in which multiple different types of features have been extracted, each feature type can be considered separately and the determination as to whether an occlusion has occurred can be made based upon a majority rule. In other embodiments, different weights can be applied to each feature type and the determination can be made based upon the results and feature weights.

With reference next to block 116, flow from this point depends upon whether there is another time segment to process. If so, flow returns to block 98 of FIG. 8A and the above-described feature extraction and comparison is repeated.

In alternative embodiments, a neural network classifier can be utilized to detect airway occlusions based upon the extracted features. Neural networks are suitable for this kind of classification because of the large number of data points that are obtained. In addition, neural networks are desirable for this application because the apnea patient population is quite large and the range of signals that can be collected is wide. Neural networks enable relatively easy training, using conventional back-propagation (BP) training algorithms. A classifier, an input layer, hidden layer, and an output layer are constructed. The output layer comprises one node, accounting for a possibility of only two classes to be classified. The number of nodes in the hidden layer is selected by an iterative training and validation scheme. Supervised learning is achieved when the features extracted from each pulse are assigned a status (either respiratory event or normal breathing) and the neural network is allowed to achieve maximum training accuracy.

In still other embodiments, airway occlusions can be detected from the extracted features using one or more of fuzzy logic, k-means clustering, and support vector machines.

As was mentioned above, the degree of occlusion can also be assessed from the ultrasonic signal. Because air attenuates the ultrasound signal, the intensity (i.e., energy) of the received ultrasound signal increases as the degree of airway occlusion increases. Therefore, that intensity can be correlated to a degree of airway occlusion.

As part of the NPSG studies, the respiratory events are routinely categorized as hypopnea (partial airway occlusion) and apnea (full airway occlusion). This categorization is clinically useful, as it guides the selection of appropriate level of pressure that is needed to keep the airway patent when patient is treated for the condition. By concurrently recording ultrasonic signals during NPSG, one can devise confidence intervals for the ratio of the means associated with hypopnea events and separate intervals for apnea events for each of the features or various ensembles of the features. The confidence intervals can be derived by conducting overnight testing of a statistically significant sample size of apnea patients in sleep laboratory with concurrent NPSG recording. The derived intervals can then serve as templates with which the mean and standard deviation ratios obtained for respiratory events are compared and events categorized.

Alternatively, when artificial neural networks or other forms of classifications are employed, the classifier can be trained using concurrently recorded NPSG results to differentiate apnea from hypopnea events. This is possible because the features described above reflect energy of the pulse (e.g., peak, area under the pulse, 25% area, etc.), which increase as the degree of occlusion increases. Hence, the degree of occlusion can be deciphered by the classifier.

As can be appreciated from the above discussion, the disclosed systems and methods provide several advantages over conventional solutions, such as NPSG. First, OSAH can be automatically and non-invasively detected without the need for NPSG, which is expensive and not readily available in all locations. In addition, airway occlusion is detected directly, as opposed indirectly as through the use of an ECG or oximeter. In addition, the degree of occlusion may be able to be identified, which is an important feature for determining the level of therapy.

The disclosed systems and methods may also be useful in evaluating the adequacy of therapy. For example, the systems and methods can be used to monitor the state of the upper airway during the application of air pressure intended to keep the airway open. In such a case, the systems and methods could be used as part of a closed-loop scheme for control of CPAP therapy to optimize the pressure applied to the airway.

While the specific application of detecting upper airway occlusion has been described in detail, it is noted that the systems and methods described herein could be applied to other applications. For example, the systems and methods could alternatively be used to detect occlusions in other segments of airways including the lungs (e.g., for asthma or chronic obstruction pulmonary disease).

1. A method for detecting airway occlusion, the method comprising:
applying ultrasonic pulses into the neck of a patient;
receiving the ultrasonic pulses after they have passed through the neck; and
analyzing the pulses to determine whether or not the airway is partially or fully occluded.

2. The method of claim 1, wherein applying ultrasonic pulses comprises applying the ultrasonic pulses into the neck using an ultrasonic transducer applied to a side of the neck.

3. The method of claim 2, wherein receiving the ultrasonic pulses comprises receiving the ultrasonic pulses using a further ultrasonic transducer applied to an opposite side of the neck.

4. The method of claim 1, wherein the applying and receiving of ultrasonic pulses is performed by a single ultrasonic transducer that is applied to the neck.

5. The method of claim 1, wherein analyzing the pulses comprises identifying and evaluating particular temporal, spatial, or spectral features of the pulses.

6. The method of claim 5, wherein the features include one or more of a peak amplitude of the pulse, the location of the peak amplitude in time, the area under the curve for the pulse, the area under the curve for the pulse associated with a fractional percentage of the peak amplitude, the time span associated with a fractional percentage of the peak amplitude, and an area under a portion of a curve of a spectrum obtained by performing Fourier transformation on the pulse.

7. The method of claim 5, wherein identifying and evaluating particular temporal, spatial, or spectral features of the pulses comprises identifying a value associated with a particular feature for each pulse of a given time segment, and calculating the mean and the standard deviation of those values.

8. The method of claim 7, wherein identifying and evaluating particular temporal, spatial, or spectral features of the pulses further comprises comparing the calculated mean and standard deviation with the mean and standard deviation of an adjacent time segment to determine how they change over time.

9. The method of claim 8, wherein comparing comprises calculating a mean ratio and a standard deviation ratio.

10. The method of claim 9, further comprising comparing the mean ratio and the standard deviation ratio with respective confidence intervals.

11. The method of claim 5, wherein analyzing the pulses further comprises inputting the features into a classifier that has been trained to identify airway occlusions.

12. The method of claim 1, further comprising quantifying the degree occlusion.

13. The method of claim 12, wherein quantifying the degree comprises determining a percentage of occlusion.

14. The method of claim 12, wherein quantifying the degree comprises calculating an apnea/hypopnea index (AHI).

15. A system for detecting airway occlusion, the system comprising:
a patient interface adapted to be placed against the neck of a patient, the interface including at least one ultrasonic transducer that is adapted to transmit or receive ultrasonic pulses; and
a control unit in communication with the patient interface, the control unit being configured to receive and store ultrasound signals collected by the patient interface.

16. The system of claim 15, wherein the patient interface includes a transmitting transducer and a receiving transducer, the transmitting transducer being positioned on one side of the interface and the receiving transducer being positioned on an opposite side of the interface.

17. The system of claim 15, wherein the patient interface further includes a pulse generator configured to generate electrical pulses that drive the ultrasonic transducer.

18. The system of claim 15, wherein the patient interface further includes a wireless transmitter configured to wirelessly transmit received ultrasonic pulses to the control unit.

19. The system of claim 15, wherein the control unit includes a pulse generator configured to generate electrical pulses that drive the ultrasonic transducer.

20. The system of claim 15, further comprising a computing device that executes a program that analyzes the ultrasound signals received and stored by the control unit to identify airway occlusions.

21. A patient interface adapted to collect ultrasound signals indicative of the patency of the upper airway, the interface comprising:
a body adapted to be placed against the neck of a patient;
at least one ultrasonic transducer mounted to the body that is adapted to transmit or receive ultrasonic pulses;
a pulse generator configured to generate electrical pulses that drive the ultrasonic transducer; and
a wireless transmitter configured to wirelessly transmit received ultrasonic pulses.

22. The patient interface of claim 21, wherein the patient interface includes a transmitting transducer and a receiving transducer, the transmitting transducer being positioned on one side of the body and the receiving transducer being positioned on an opposite side of the body.

23. The patient interface of claim 22, wherein the patient interface includes multiple transmitting transducers and multiple receiving transducers, the transmitting transducers being positioned on one side of the body and the receiving transducers being positioned on an opposite side of the body.

24. A non-transitory computer-readable medium that stores a sleep disordered breathing diagnosis system comprising:
logic configured to receive an ultrasound signal based upon data collected from a patient; and
logic configured to analyze ultrasonic pulses of the ultrasound signal to determine whether or not an airway of the patient is partially or fully occluded.

25. The computer-readable medium of claim 24, wherein the logic configured to analyze comprises logic configured to select a segment of the ultrasound signal to process and logic configured to identify and evaluate a particular temporal, spatial, or spectral feature of each pulse of the segment.

26. The computer-readable medium of claim 25, wherein the feature is one of a peak amplitude of the pulse, the location of the peak amplitude in time, the area under the curve for the pulse, the area under the curve for the pulse associated with a fractional percentage of the peak amplitude, the time span associated with a fractional percentage of the peak amplitude, and an area under a portion of a curve of a spectrum obtained by performing Fourier transformation on the pulse.

27. The computer-readable medium of claim 25, wherein the logic configured to identify and evaluate comprises logic configured to identify a value associated with the feature for each pulse of the time segment and to calculate the mean and the standard deviation of those values.

28. The computer-readable medium of claim 24, wherein the logic configured to identify and evaluate further comprises logic configured to compare the calculated mean and standard deviation with the mean and standard deviation of an adjacent time segment to determine how they change over time.

29. The computer-readable medium of claim 28, wherein the logic configured to compare comprises logic configured to calculate a mean ratio and a standard deviation ratio.

30. The computer-readable medium of claim 29, further comprising logic configured to compare the mean ratio and the standard deviation ratio with respective confidence intervals.

31. The computer-readable medium of claim 25, wherein the logic configured to analyze the pulses comprises a classifier that has been trained to identify airway occlusions.
